(19) United States
(12) Patent Application Publication
(10) Pub. No.: US 2025/0152295 A1
(43) Pub. Date: May 15, 2025

(54) PRIORITIZATION OF MULTIPLE OBJECTS IN AN ASSISTANCE FUNCTIONALITY FOR A SURGICAL MICROSCOPY SYSTEM

(71) Applicant: CARL ZEISS MEDITEC AG, Jena (DE)

(72) Inventors: Markus PHILIPP, Stuttgart (DE); Stefan SAUR, Aalen (DE); Neal BACHER, Stuttgart (DE)

(21) Appl. No.: 18/945,685

(22) Filed: Nov. 13, 2024

(30) Foreign Application Priority Data
Nov. 15, 2023 (DE) 10 2023 131 861.6

(51) Int. Cl. A61B 90/25; A61B 34/20

(52) U.S. Cl. CPC A61B 90/25; A61B 34/20

(57) ABSTRACT

A description is given of techniques in connection with carrying out an assistance functionality for a surgical microscopy system. The assistance functionality—for example auto-centring or measurement of a surgical instrument—is carried out in connection with multiple objects that are depicted in an image captured by way of the surgical microscopy system. Prioritization information for the objects is furthermore also taken into consideration, for example in order to perform classification into relevant and irrelevant objects.

### TECHNICAL FIELD

Various examples of the disclosure relate to techniques for carrying out an assistance functionality for a surgical microscopy system. Various examples relate in particular to prioritization between different objects in connection with such an assistance functionality.

### BACKGROUND

Medical surgical microscopy systems (also referred to as robotic visualization systems or surgical visualization systems) having a robotic stand for positioning a microscope are known from the prior art; see for example DE 10 2022 100 626 A1.

The robotic stand is able to be controlled manually. However, there are also known techniques in which the robotic stand is controlled automatically, for example in order to enable auto-centring and/or auto-focusing on a particular object, such as for example a surgical instrument (also referred to as surgical equipment or surgical tool or operating tool). A user command in this case triggers a positioning procedure in which the robotic stand and/or an objective optical unit of the microscope are driven. Such techniques are for example known from U.S. Pat. No. 10,456,035 B2.

It has been observed that, in particular in relatively complex surgical scenes, for example containing a large number of surgical instruments and/or surgical instruments of different types, such techniques from the prior art may deliver undesirable results. By way of example, positioning is sometimes carried out on an incorrect surgical instrument that was not meant by the surgeon at all.

In order to remedy such disadvantages, U.S. Pat. No. 10,769,443 B2, for example, discloses recognizing dominant surgical instruments from a set of visible surgical instruments. Such techniques also have certain disadvantages and limitations. By way of example, it has been found that the techniques described in that document do not work well for some types of surgical instruments. This means that, depending on the type of surgical instrument, poor results are achieved. Due to the large number of surgical instruments available, this weakens the acceptance of the system, and unexpected behaviour may occur.

### SUMMARY

There is therefore a need for improved techniques for assistance functionalities for surgical microscopy systems. There is in particular a need for techniques that enable robust classification of the priority of imaged objects to which the assistance functionality is able to relate. There is a need to classify objects as dominant or non-dominant.

This object is achieved by the features of the independent claims. The features of the dependent claims define embodiments.

Various examples are based on the finding that techniques for recognizing dominant surgical instruments, as are known in the prior art, exhibit low robustness to variations in the appearance of the surgical instruments. This is due to the fact that a particularly large number of different types of surgical instruments exist in different operating environments, for example in the three-digit range. Various examples are based on the finding that it is difficult, using an algorithm, to distinguish robustly between such a large number of possible result classes within the scope of a surgical instrument type classification. By way of example, when using machine-learned classification models, an out-of-distribution situation may often occur in which the appearance of a specific type of surgical instrument was not taken into consideration in the training of the machine-learned classification model. The model may then deliver unpredictable results.

In addition, various examples are based on the finding that techniques for recognizing dominant surgical instruments, as are known in the prior art, ignore the fact that a certain type of surgical instrument may be dominant in one context and might not be dominant in another context. It has been recognized that considering the context may be helpful in prioritization.

A description is given below of aspects in connection with a surgical microscopy system having a robotic stand and a microscope carried by the robotic stand. A description is given in particular of techniques in connection with an assistance functionality for assisting a surgeon. The assistance functionality is carried out with respect to at least one object that is depicted in a corresponding image (for example captured by a microscope camera or an environment camera). Examples of such assistance functionalities are auto-positioning, auto-centring, auto-orientation, auto-zoom or auto-focus with respect to one or more objects; further examples comprise, for example, the measurement of one or more objects.

Techniques for separating relevant and irrelevant objects from one another are disclosed. Relevant objects are sometimes also referred to as dominant objects.

More generally speaking, a description is given of techniques as to how to be able to determine prioritization information for the various objects. The assistance functionality may then be carried out taking into consideration the prioritization information. By way of example, the assistance functionality may take into consideration only those objects that are classified as relevant (that is to say high-priority) based on the prioritization information. Gradual determination of corresponding prioritization information may also be used, so that higher-priority objects are taken into consideration to a greater extent when carrying out the assistance functionality.

The determination of the prioritization information may, generally speaking, correspond to a regression task or a classification task.

The prioritization information is determined, according to various disclosed variants, based on movement information for the various objects. The use of movement information has particular advantages in relation to reference implementations in which a type classification of the objects (for example an instance segmentation with associated classification of the instances) has to be carried out. Robust prioritization may in particular be achieved even for a large number of different, a priori unknown scenes. By way of example, it is not necessary to parameterize a classification model for determining the type of the objects. By way of example, it is not necessary to train any corresponding machine-learned classification model, which makes it possible to dispense with complex training campaigns for collecting images of different types of objects. The prioritization information may be obtained without any classification and, in particular, without any instance segmentation of the objects.

Nevertheless, in some variants, it is conceivable for further information to be taken into consideration when determining the prioritization information, for example semantic context information in relation to the imaged scene, or else whether a particular surgical instrument is carried in the left or right hand.

A computer-implemented method for controlling a surgical microscopy system is disclosed. The surgical microscopy system comprises a stand. The surgical microscopy system also comprises a microscope. The microscope is carried by the stand.

By way of example, the stand may be a robotic stand or a partially robotic stand.

The method comprises driving a camera of the surgical microscopy system. By way of example, a microscope camera and an environment camera could be driven. A sequence of images is obtained by driving the camera. By way of example, the sequence of images may thus correspond to a time sequence of images depicting a scene.

The method also comprises determining movement information. The movement information is determined for each of two or more objects depicted in the sequence of images. The movement information is determined based on the sequence of images.

By way of example, heuristic or machine-learned models may be used to determine the movement information. By way of example, threshold value comparisons could be carried out between one or more variables indicated by the movement information and one or more predefined threshold values.

By way of example, the movement information may specify an optical flow. As an alternative or in addition, the movement information may specify activity regions, that is to say regions in the sequence of images in which there is a relatively large change in contrast between the images of the sequence. It would be conceivable for the movement information to specify movement patterns for the objects. By way of example, the movement information may specify a movement amplitude and/or movement frequencies for the objects. Combinations of such contents of the movement information as described above are also conceivable.

It would be conceivable (but not necessary) for the movement information to be linked to a positioning of the objects in the sequence of the images. By way of example, it would be possible to first localize the objects in the sequence of the images and then to determine corresponding movement information for each of the localized objects.

The method furthermore comprises determining prioritization information for the two or more objects; this is carried out based on the movement information. By way of example, the prioritization information specifies which objects are dominant or relevant and which objects are not dominant or irrelevant. In addition to such binary class assignment of the various objects as part of the prioritization information, a multi-dimensional class assignment or regression would also be conceivable. By way of example, a prioritization value could be output, for example in the range from 0 (=irrelevant) to 10 (=relevant).

Based on the prioritization information, an assistance functionality is then carried out in connection with the two or more objects. This may mean for example that objects that have a larger (smaller) prioritization are taken into consideration to a greater (lesser) extent as part of the assistance functionality.

As part of the assistance functionality, for example, the robotic stand could be driven, for example in order to perform auto-positioning with respect to a reference point determined based on one or more objects. As part of the assistance functionality, the appearance of one or more objects in one or more images, for example a microscope image, may be evaluated.

A data processing device is disclosed. The data processing device is configured to control a surgical microscopy system. The data processing device comprises a processor. The processor is configured to load program code from a memory and to execute it. Executing the program code causes the processor to carry out the method described above for controlling a surgical microscopy system.

A surgical microscopy system comprising such a data processing device is also disclosed.

The features set out above and features that are described hereinbelow may be used not only in the corresponding combinations explicitly set out, but also in further combinations or in isolation, without departing from the scope of protection of the present invention.

### BRIEF DESCRIPTION OF THE FIGURES

FIG. 1 schematically illustrates a surgical microscopy system in accordance with various examples.

FIG. 2 schematically illustrates different fields of view in connection with a microscope and an environment camera of an exemplary surgical microscopy system.

FIG. 3 is a flowchart of one exemplary method.

FIG. 4 is a flowchart of one exemplary method.

FIG. 5 illustrates an image, captured by way of a camera, in which multiple surgical instruments are visible.

FIG. 6 corresponds to FIG. 5, with prioritization information for the various surgical instruments additionally being displayed.

### DETAILED DESCRIPTION

The properties, features and advantages of this invention described above and the way in which they are achieved will become clearer and more clearly understood in connection with the following description of the exemplary embodiments that are explained in greater detail in connection with the drawings.

The present invention is explained in greater detail below on the basis of preferred embodiments with reference to the drawings. In the figures, identical reference signs denote identical or similar elements. The figures are schematic representations of various embodiments of the invention. Elements illustrated in the figures are not necessarily illustrated as true to scale. Rather, the various elements illustrated in the figures are rendered in such a way that their function and general purpose become comprehensible to a person skilled in the art. Connections and couplings between functional units and elements illustrated in the figures may also be implemented as an indirect connection or coupling. A connection or coupling may be implemented in a wired or wireless manner. Functional units may be implemented as hardware, software or a combination of hardware and software.

Techniques in connection with the operation of surgical microscopy systems are described below. The described techniques make it possible to carry out an assistance functionality, for example automatic configuration of one or more components of the surgical microscopy system. In general, the assistance functionality is implemented with respect to at least one object from a multiplicity of objects that are visible in an image captured by way of the surgical microscopy system.

According to various examples, prioritization information is determined for the objects. The prioritization information is determined based on movement information for the objects.

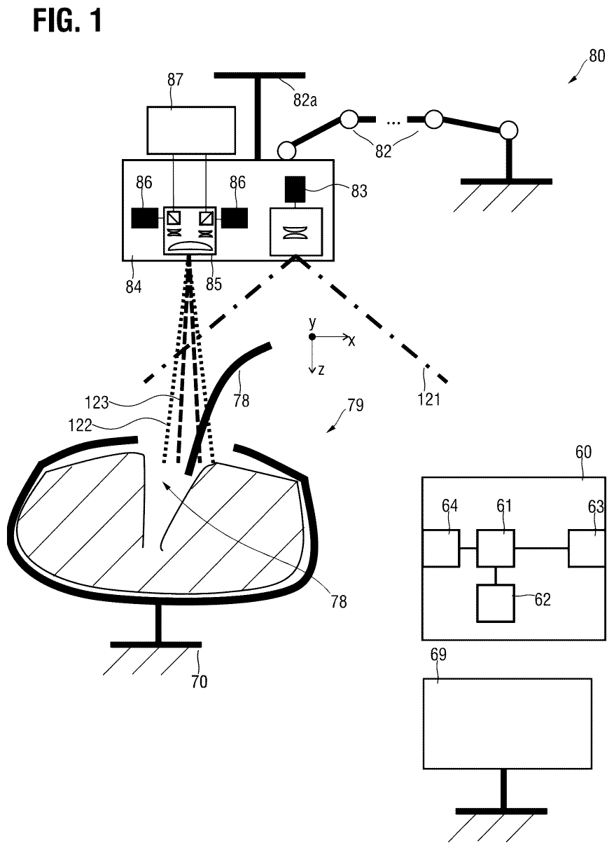

FIG. 1 schematically illustrates aspects with respect to an exemplary surgical microscopy system 80. The surgical microscopy system 80 is used for microscopic imaging of an examination region during a surgical intervention. For this purpose, a patient 79 is placed on an operating table 70. The situs 78 is shown with a surgical instrument 78.

The surgical microscopy system 80 includes a robotic stand 82, which carries a positionable head part 81. The robotic stand 82 may have different degrees of freedom, depending on the variant. Robotic stands 82 having six degrees of freedom for positioning the head part 81, that is to say translation along each of the x-axis, y-axis and z-axis and rotation about each of the x-axis, y-axis and z-axis, are known.

The robotic stand 82 may, as shown in FIG. 1, have a handle 82a.

While FIG. 1 discusses a robotic stand 82, it would also be generally possible to use an only partially robotic or manual stand.

The head part 81 comprises a microscope 84 having optical components 85, such as for example an illumination optical unit, an objective optical unit, a zoom optical unit, etc. The microscope 84 furthermore comprises, in the illustrated example, a microscope camera 86 (here a stereo camera with two channels; a mono optical unit would however also be conceivable) using which images of the examination region are able to be captured and are able to be reproduced for example on a screen 69. The microscope 84 is therefore also referred to as a digital microscope. A field of view 123 of the microscope camera 86 is also shown.

In the example of FIG. 1, the microscope 84 also comprises an eyepiece 87 having an associated field of view 122. The eyepiece 87 is thus optional. By way of example, the detection beam path may be split by way of a beam splitter, such that both an image is able to be captured by the camera 86 and observation through the eyepiece 87 is possible. It is not necessary in all variants for the microscope 84 to have an eyepiece. Purely digital microscopes 84, in which no eyepiece is present, are also possible.

In the example of FIG. 1, the head part 81, carried by the stand 82, of the surgical microscopy system 80 also comprises an environment camera 83. The environment camera 83 is optional. While the environment camera 83 in the example of FIG. 1 is illustrated as being integrated into the microscope 84, it would be possible for it to be arranged separately from the microscope 84. By way of example, the environment camera may be a CCD camera. The environment camera may also have depth resolution. As an alternative or in addition to the environment camera, it would also be conceivable for other assistive sensors to be present, for example a distance sensor (for instance a time-of-flight camera or an ultrasonic sensor or a sensor with structured illumination). A field of view 121 of the environment camera 83 is shown in FIG. 1.

The surgeon thus has multiple options for observing the examination region: using the eyepiece 87, using the microscope image taken by the camera 86, or using an overview image taken by an environment camera 83. The surgeon may also observe the examination region directly (without magnification).

Next, aspects in relation to the fields of view 121, 122 and 123 will be discussed.

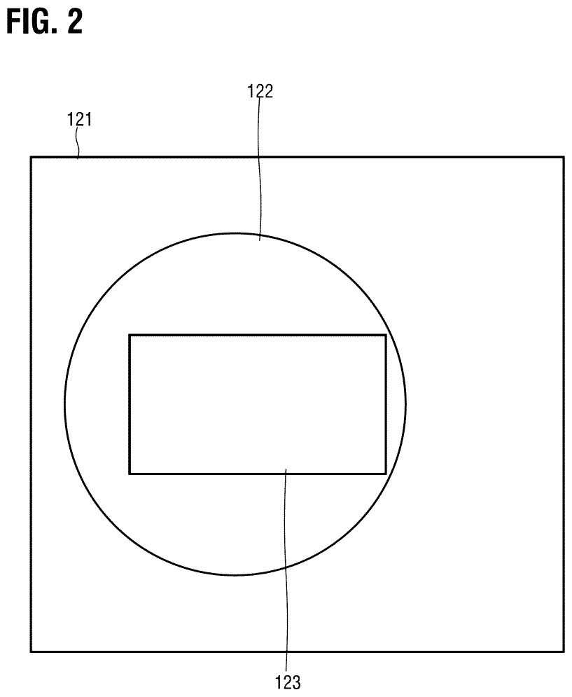

FIG. 2 illustrates aspects in relation to the various fields of view. FIG. 2 illustrates the field of view 121 of an environment camera of the surgical microscopy system 80. Overview images thus depict a relatively large region. In addition, the field of view 122 of the eyepiece and the field of view 123 of the microscope camera 86 are illustrated by way of example. The fields of view 121, 122, 123 do not have to be arranged in a manner centred on one another.

Referring once again to FIG. 1: The various components of the surgical microscopy system 80, such as for example the robotic stand 82, the microscope 84 or the one or more further components, such as for example the environment camera 83, are controlled by a processor 61 of a data processing device 60.

The processor 61 may be designed for example as a general central processor (CPU) and/or as a field-programmable logic module (FPGA) and/or as an application-specific integrated circuit (ASIC). The processor 61 is able to load program code from a memory 62 and execute it.

The processor 61 is able to communicate with various components of the surgical microscopy system 80 via a communication interface 64. By way of example, the processor 61 may drive the stand 82 so as to move the head part 81 with respect to the operating table 70, for example in translation and/or in rotation. The processor 71 may for example drive optical components 85 of the microscope 84 in order to change a zoom and/or focus (focal length). Images from the environment camera, where present, could be read out and evaluated. In general, images may be evaluated and an assistance functionality may be carried out based on the evaluation.

The data processing device 60 furthermore comprises a user interface 63. The user interface 63 may be used to receive commands from a surgeon or generally from a user of the surgical microscopy system 80. The user interface 63 may have different configurations. By way of example, the user interface 63 may comprise one or more of the following components: handles on the head part 81; foot switch; voice input; input via a graphical user interface; etc. It would be possible for the user interface 63 to provide a graphical interaction via menus and buttons on the monitor 69.

A description is given below of techniques as to how it is possible, by way of the surgical microscopy system 80, to prepare an assistance functionality. The assistance functionality may be requested for example by a user command.

As a general rule, such a user command may take different forms. By way of example, the user command could specifically identify a particular object. By way of example, the user could specify, by voice command: “Auto-centring on the surgical instrument 1”. Such a user command thus specifies exactly the object with respect to which the surgical microscopy system 80 is to be configured (here with respect to the “surgical instrument 1”). In other examples, however, it would also be conceivable for the user command to be non-specific for a particular object. By way of example, the user could specify, by voice command or by pressing a button: “Auto-centring”. The object with respect to which the auto-centring should be carried out is thus not specified (even if multiple objects that are candidates for the auto-centring are visible). The user command is not unambiguous in this example. The user command does not distinguish between multiple visible surgical instruments. In reference implementations, the user command may then be misinterpreted and for example auto-centring may be performed with respect to the incorrect surgical instrument. The user expectation (for example auto-centring on “surgical instrument 1”) might then not correspond to the actual system behaviour (for example auto-centring on the geometric centre of all visible surgical instruments).

A description is given below of techniques that make it possible, in connection with an assistance functionality triggered by a user command, to better meet the user expectation than in reference implementations. A deterministic system behaviour is made possible, this delivering reproducible and comprehensible results in a wide variety of situations and/or in the face of a wide variety of scenes. Such techniques are based on the finding that, in particular in high-stress situations under time pressure, as typically occur in a surgical environment, it is necessary for the system behaviour of an automatic control system to match the user expectation exactly.

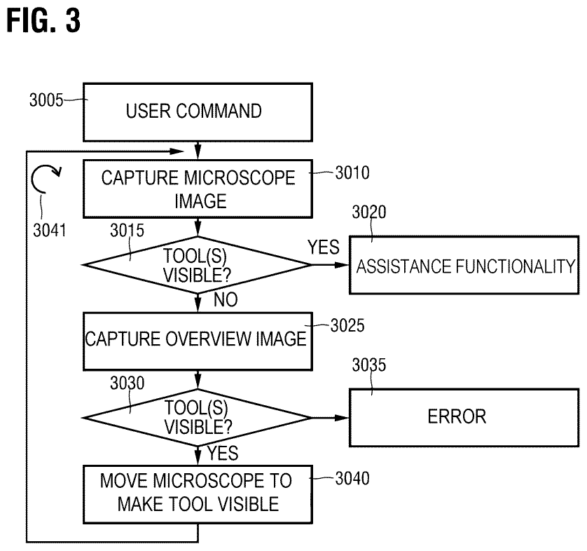

FIG. 3 is a flowchart of one exemplary method. The method of FIG. 3 relates to techniques in connection with the configuration of a surgical microscopy system for imaging an object in response to a user command.

The method from FIG. 3 may be carried out by a processor of a data processing device, for example by the processor 61 of the data processing device 60 of the surgical microscopy system 80 from the example of FIG. 1. The processor is able, for this purpose, to load program code from a memory and then execute it.

A user command is received in box 3005. The user command may request an assistance functionality. By way of example, the user command requests that the microscope camera be configured to image an object, for example a surgical instrument, implicitly or explicitly. The user command is received from a user interface. By way of example, the user command may request auto-alignment or auto-focusing. The user command could request the measurement of a surgical instrument.

The user command may be non-specific for a particular object. The user command might not specify to which of multiple visible objects it relates.

A microscope image is captured in box 3010 (optional box). For this purpose, the microscope camera of the microscope is driven; cf. microscope camera 87 of the microscope 84 in the example of FIG. 1. As was already discussed above in connection with FIG. 2, the microscope camera typically has a comparatively small field of view, namely in particular a field of view that is smaller than the field of view of an eyepiece or an environment camera (where present).

In box 3015 (optional box), the object identified by the user command, for example a surgical instrument, is then searched for in the microscope image from box 3010. If the object is already positioned in the field of view of the microscope camera, the object is found in box 3015, that is to say it is visible in the microscope image: box 3020 is then carried out. This involves carrying out an assistance functionality based on the microscope image (for example auto-centring or auto-focusing or measurement of the object).

Scenarios may occur in which the object is not found in the microscope image in box 3015. This means that the object is not positioned in the central region of the scene that is imaged by the microscope camera. In such a case, in box 3025, the environment camera is driven so as to capture an overview image. This is done to check whether the object is positioned in the peripheral region of the scene that is imaged by the environment camera, but not by the microscope camera.

In box 3030, it is then possible to determine whether the object is visible in the overview image. In box 3030, it is possible to determine whether the object is located in the peripheral region, that is to say in the region of the scene that is covered by the field of view of the environment camera, but not by the field of view of the microscope (in FIG. 2, this is the region that lies outside the field of view 123 but within the field of view 121).

Generally speaking, the object is thus searched for in the overview image. If the object is not found in the overview image, an error is output in box 3035. Otherwise, box 3040 is carried out.

In box 3040, a control command is provided for the robotic stand to move the microscope such that the object is positioned in the field of view of the microscope camera, that is to say in a central region of the scene. This means that, in box 3040, a rough alignment is carried out, such that the surgical instrument is then also visible in a microscope image that is captured in another iteration 3041 of box 3010.

In summary, it is thus possible, by way of the method in FIG. 3, to first capture an overview image in order to localize surgical instruments for assistive functions using the environment camera. In this case, this overview image from the environment camera is first evaluated, and it is determined whether it contains a surgical instrument. The robotic stand is then driven so as to move the recognized surgical instrument into the field of view of the microscope camera. The actual assistance functionality may then be performed based on an evaluation of one or more microscope images.

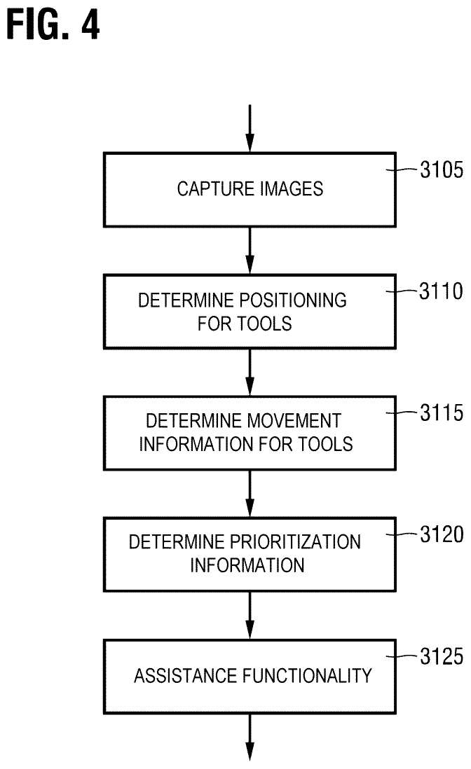

It may sometimes be the case that the user command from box 3005 does not specify exactly the at least one object in relation to which the assistance functionality in box 3020 is supposed to be carried out. By way of example, four surgical instruments could be visible, these all being candidates for the assistance functionality (for example auto-centring). It is therefore initially unclear with respect to which subset of the visible surgical instruments a search may have to be carried out in box 3030 followed by positioning in box 3040. In order to solve such problems, it is possible in particular to apply techniques as described below in connection with FIG. 4.

FIG. 4 is a flowchart of one exemplary method. The method of FIG. 4 concerns techniques for preparing to carry out an assistance functionality. The assistance functionality may for example use the position and/or orientation (positioning; also referred to as pose or absolute position) and/or other geometric properties of surgical instruments—even if a large number of such surgical instruments are visible in the corresponding images, for example overview images or microscope images. As an alternative or in addition, it would be conceivable for the assistance functionality to take into consideration movement information determined based on a sequence of images.

The techniques of FIG. 4 concern aspects for avoiding any ambiguities due to the large number of corresponding visible surgical instruments.

Aspects of FIG. 4 may be used for example in connection with the localization of surgical instruments in box 3030, 3040 from FIG. 3. Aspects of FIG. 4 may, as an alternative or in addition, be used in connection with the assistance functionality in box 3020 from FIG. 3. FIG. 4 may however also be implemented on a standalone basis, that is to say not in the context of FIG. 3.

The various variants in FIG. 4 are described in connection with an implementation of objects in the form of surgical instruments. However, corresponding techniques could also be performed for other types of objects.

Examples of surgical instruments are in general: scalpel, forceps, scissors, needle holder, clamp, aspirator, trocar, coagulator, electrocauter, retractor, drill, spreader, osteotome, suture material, knot pusher, periosteal elevator, haemostat, lancet, drainage, thread cutter, spatula, ultrasonic aspirators.

The method of FIG. 4 may be carried out by a processor of a data processing device, for example by the processor 61 of the data processing device 60 of the surgical microscopy system 80 from the example of FIG. 1. The processor is able, for this purpose, to load program code from a memory and execute the program code.

In box 3105, a sequence of images is captured. This may be triggered for example by a user command, for example as described in connection with box 3005 from FIG. 3.

The method from FIG. 4 may for example be triggered by a user command that requests a particular assistance functionality. By way of example, the user could request auto-alignment of the field of view of the microscope camera and/or auto-focusing. As a general rule, it would be conceivable for the user command not to specify the specific object on which the assistance functionality is to be based or that defines said assistance functionality. This thus means that it is possible to receive a user command that requests the assistance functionality in relation to a non-specific one of the displayed objects. The user command may thus contain an ambiguity with respect to the surgical instruments to be taken into consideration.

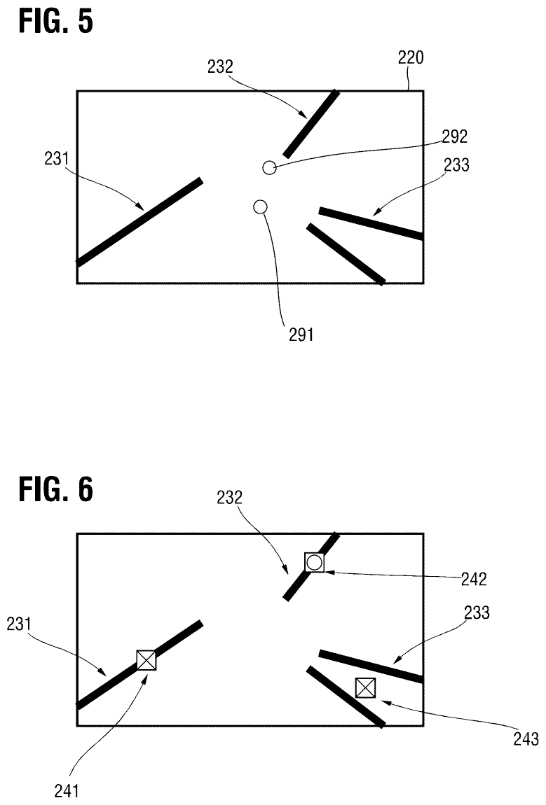

In box 3105, a camera, for example a microscope camera or an environment camera, is driven. The images depict a surgical scene over a particular period of time. One example of an image 220 is illustrated in FIG. 5. In FIG. 5, it may be seen that a total of three surgical instruments 231, 232, 233 are visible.

Referring once again to FIG. 4: In the optional box 3110, the visible surgical instruments are localized and their orientation is optionally determined. Positioning information may thus be determined in box 3110. Such positioning information may comprise a localization of the surgical instruments in the one or more images from box 3105. Such a positioning may comprise an orientation of the surgical instruments in the two or more images from box 3105. The positioning information may be obtained for example by way of point localization or by way of bounding boxes. Segmentation of objects of surgical instruments in the images could also be performed. An instance segmentation could be performed.

The positioning may be ascertained for example based on an optical flow. By way of example, it is possible to use techniques as disclosed in connection with WO 2022/161930 A1.

The positioning may also optionally be carried out in a reference coordinate system. By way of example, based on the positioning of surgical instruments in the images from box 3105, when the pose of the corresponding camera and the imaging properties of the camera are known, it is possible to infer an absolute positioning of the surgical instruments in a reference coordinate system. Such a technique may be used in particular in connection with images captured by way of an environment camera.

Then, box 3115 comprises determining movement information for the surgical instruments from the sequence of the images. It would be conceivable, but not necessary, for the movement information to be determined based on the positioning information from box 3110. As already explained above, box 3110 is optional and it is possible that the positioning information is not needed to determine the movement information. By way of example, the movement information may also be determined without prior localization, for example based on the optical flow between two successively captured images. Details regarding the determination of the movement information will be explained later.

Prioritization information is then determined in box 3120 based on the movement information.

Based on the positioning from box 3110 and/or the movement information from box 3115, and based on the prioritization information from box 3120, an assistance functionality is then carried out (in box 3125). By way of example, auto-alignment and in particular auto-centring on the activity centre of surgical instruments may be carried out. For this purpose, for example, one or more activity regions may be determined from the movement information and their geometric centre or geometric centroid may be used as activity centre. Auto-alignment and in particular auto-centring on a particular surgical instrument or a geometric centre of multiple surgical instruments could be carried out. Auto-focusing on a particular surgical instrument, for example on the tip thereof, could be carried out.

The assistance functionality relates to the surgical instruments, for example to the positioning information or else to other geometric properties of the surgical instruments (for example distance measurement between surgical instruments or opening angle of a stapling instrument, etc.).

Various examples are based on the finding that it may be helpful, in certain variants, if the assistance functionality takes into consideration only a subset of all of the surgical instruments visible in the corresponding image or, more generally, is carried out based on the prioritization information from box 3110. This means for example that the positioning of and/or movement information in relation to a particular surgical instrument of the visible surgical instruments is taken into consideration to a greater extent than the positioning of and/or movement information in relation to another surgical instrument of the visible surgical instruments (whose positioning and movement information may also not be taken into consideration at all).

In other words and more generally, a distinction may thus be made between more relevant and less relevant surgical instruments; more relevant surgical instruments are then taken into consideration to a greater extent in the assistance functionality than less relevant surgical instruments.

This is explained with reference to the example of FIG. 5 in one specific example: The exemplary scene of FIG. 5 contains an aspirator 231 (purpose: aspirate blood), a bipolar coagulator 233 (purpose: stop bleeding) and a retractor 232 (purpose: hold back brain tissue). For the surgeon, the relevant instruments are the aspirator and the bipolar coagulator, as they perform the primary surgical action in the image shown (stop bleeding, aspiration). However, the retractor is not relevant in this scene: The retractor passively holds back brain tissue, is spatially fixed, and does not perform any primary surgical action. This is just one example of prioritizing relevant surgical instruments. As an alternative, instruments of the assistant surgeon may also be visible in the image, but be of only little relevance to the chief surgeon. In the example of FIG. 5, the geometric centroid of the surgically relevant instruments should now be determined, and auto-centring on this geometric centroid should then be performed. The geometric centroid 291 of only the surgically relevant instruments (that is to say without the retractor 232) is spaced from the geometric centroid 292 of all instruments 231, 232, 233. From the user's point of view, if the instruments 231, 233 are prioritized incorrectly or not prioritized in relation to the surgical instrument 232, then poor auto-centring may occur because the centroid 292 is then centred instead of the centroid 291. A corresponding scenario may also be described in connection with auto-focusing. In comparison to the variant outlined above, in which auto-centring is carried out on the geometric centroid 291, it may be advantageous to perform auto-focusing on the tip or another characteristic position of the highest-priority instrument (that is to say for example on the surgical instrument 231). This is based on the finding that the surgeon typically operates primarily using a single instrument and other instruments are of secondary significance behind this primary instrument.

Various examples are based on the finding that it is particularly easily possible to distinguish between relevant and less relevant surgical instruments based on the movement information. In other words, it is reliably possible to determine prioritization information based on the movement information.

Various implementations of the movement information are conceivable, and some examples are discussed below. These examples may also be combined with one another.

By way of example, the movement information may comprise an optical flow between successive images of the sequence of images. The movement information may specify one or more activity regions. Corresponding techniques are disclosed in detail in WO2022161930 A1, the disclosure content of which is incorporated herein by cross-reference.

By way of example, it would be conceivable for the movement information to be indicative of a time-averaged movement magnitude of the movement of each individual object of the objects. Such a movement magnitude may be determined for example such that multiple objects are localized (cf. box 3110), and then the movement of the object is traced/tracked in each case within a corresponding region in which the corresponding object is localized. Generally speaking, the movement information may thus be determined based on a positioning of the objects.

By way of example, the movement information may be indicative of one or more movement patterns of the objects. By way of example, it is conceivable for a particular type of surgical tool—for example an aspirator—to preferably be used in a circular movement; such a movement pattern (circular movement) may then be recognized, and it may for example be inferred therefrom that the aspirator is a low-priority auxiliary tool. On the other hand, another type of surgical tool, for example a scalpel, could be moved primarily in translation, that is to say moved back and forth.

Such a movement pattern (translational movement between two endpoints) may then be recognized and it may be inferred for example that the scalpel is a high-priority primary tool. In general, recurrent movement patterns predefined by the surgical use of a particular tool may be taken into consideration when prioritizing a tool. Such movements of the tool are used for the application of the tool in the surgical procedure itself (for example, in the above example of the aspirator for aspirating blood or in connection with the above example of the scalpel for cutting tissue); they are thus not movements that are carried out as part of specific gesture recognition and have no purpose in themselves beyond carrying out the gesture.

Further examples of movement information would be for example information regarding the direction distribution of a movement of the corresponding object. It would also be conceivable to specify the movement frequencies of the movement of the object.

Various exemplary implementations of the movement information have been disclosed above. Combinations of such disclosed variants of the movement information may also be used in the various techniques described herein.

There are various possibilities for implementing the prioritization information. By way of example, a segmentation map could be output that, with respect to one of the captured images, classifies the regions of the image in which the objects are displayed into different priority classes. It would also be conceivable for labels to be output, these being arranged at object positions. These labels may then display the priority information. It would thus be conceivable for the prioritization information to comprise a localization (for example point localization, bounding box, etc.) with a prioritization label. A corresponding example is illustrated in FIG. 6 for a point localization. Said figure shows a respective corresponding label 241, 242, 243 for the surgical instruments 231, 232, 233 at a centre position of the surgical instruments 231, 232, 233. These labels 241-243 indicate the priority of the surgical instruments, wherein the labels 241, 243 indicate a high priority; and the label 242 indicates a low priority. By way of example, the prioritization information could indicate activity centres for relevant and irrelevant objects. Generally speaking, the prioritization information may thus be linked to the positioning of the various objects.

The prioritization information may either assume a continuous value (for example from 1=low priority; to 10=high priority; regression) or else a class assignment of the objects to predefined classes; for example, a class assignment to a first class and a second class would be possible (and optionally one or more further classes). The class assignment may be binary. By way of example, the first class may concern relevant objects and the second class may comprise irrelevant objects. It is then possible for the assistance functionality to be carried out based solely on the positioning of those one or more objects that are assigned to the first class (that is to say those objects that are assigned to the second class are ignored). By way of example, in connection with FIG. 6, a scenario has been discussed in which the surgical instruments 231, 233 are assigned to the first class; and the surgical instrument 232 is assigned to the second, irrelevant class.

The prioritization information may be taken into consideration in various ways in connection with the assistance functionality. A few examples are explained hereinafter.

The assistance functionality may for example concern the measurement of a surgical instrument. It is then possible for example to measure the surgical instrument that has the highest priority.

The assistance functionality may also comprise automatic configuration of one or more components of the surgical microscopy system. As part of the assistance functionality, a target configuration may then be determined for one or more components of the surgical microscopy system. When determining the target configuration, the positioning of those of the two or more objects that are prioritized higher based on the prioritization information may be taken into consideration to a greater extent compared to the positioning of those of the two or more objects that are prioritized lower based on the prioritization information. A sliding transition between consideration to a greater and lesser extent would be conceivable, as would a binary transition, that is to say the positioning of higher-priority objects is taken into consideration and the positioning of lower-priority objects is not taken into consideration. Different target configurations are determined depending on the assistance functionality. By way of example, the target configuration of the surgical microscopy system could comprise an alignment of a field of view of the microscope of the surgical microscopy system (for example a microscope camera or an eyepiece) with respect to at least one of the two or more surgical instruments that is selected on the basis (dependent on) of the prioritization information. This thus means for example that the centroid of all higher-priority surgical instruments is determined based on the corresponding positions, and the robotic stand is then driven such that the centroid lies in the centre of the field of view of the microscope, as already explained above in connection with FIG. 5. However, this is just an example. It would also be conceivable to select only a single surgical instrument from the multiplicity of visible surgical instruments—namely the one with the highest priority—and for the field of view of the microscope to be auto-arranged at a relevant point of this selected surgical instrument, for example at the tip thereof. In a further variant, the target configuration of the surgical microscopy system comprises auto-focusing taking into consideration multiple surgical instruments, wherein prioritization is carried out within the surgical instruments. It would thus be possible, in a further variant, for the target configuration of the surgical microscopy system to comprise focusing of the microscope with respect to one of the two or more predefined surgical instruments that is selected on the basis of the prioritization information. It is thus possible to provide an auto-focus in relation to a high-priority surgical instrument. When focusing on a geometric centroid of two or more surgical instruments (or objects in general), then the depth centroid may also be taken into consideration for the auto-focusing, in addition to the geometric centroid. This thus means that depth is determined for all objects that are incorporated into the determination of the local centroid; and focus is then placed on the mean value. This should be distinguished from an (in principle also possible) variant in which the focus is placed on the depth at the local centroid.

Next, details are disclosed as to how the mapping of movement information to prioritization information may look. In other words, a description is given below as to how exactly the prioritization information is able to be determined based on the movement information in box 3120.

In the first example, the prioritization information is determined based on the movement information using a predefined criterion. This thus means, in other words, that the criterion used to determine the prioritization information based on the movement information is independent of the movement information itself. By way of example, a fixed threshold value could be used, this being compared with a value of the movement information. A lookup table could be used, this mapping different values of the movement information to different prioritization information. A fixed, predefined function could be used, this converting movement information into prioritization information. As an example, the movement magnitude (quantified by the optical flow, for example) could for example be taken into consideration. This movement magnitude could be compared with a predefined threshold value. If the movement magnitude is greater than the predefined threshold value, then the corresponding surgical instrument is assigned to a first, relevant class; otherwise to a second, irrelevant class. With reference to the example of FIG. 5: in said figure, the movement magnitude indicates that the aspirator 231 and the bipolar coagulator 233 are moved significantly (that is to say the movement magnitude is greater than a threshold value); but the retractor 232 is not. Namely, the retractor 232 is fixed to the patient and only moves slightly with the brain tissue. In this case, the surgical instruments 231, 233 are classified in the high-priority class and the surgical instrument 232 is classified in a low-priority class. In general, fixed movement threshold values may be used, including for differently defined movement information. Such a criterion, which is predefined with respect to the movement information, may be set by a user. By way of example, different users may have different preferences regarding the classification of surgical instruments as relevant and irrelevant (that is to say regarding the classification of surgical instruments as being associated with high priority or low priority). However, it would also be conceivable for such a criterion to be fixedly preprogrammed and not changed by the user.

In the second example (as an alternative or in addition to the first example above), the prioritization information is determined based on the movement information using a relative criterion. The relative criterion is ascertained based on the movement information. This means, in other words, that for example a relative ratio of values of the movement information ascertained for different surgical instruments is taken into consideration; or a threshold value that is adjusted based on the values of the movement information (for example at 50% of the maximum, etc.). By way of example, a ranking of values of the movement information may be taken into consideration. By way of example, only a single object could be included in a first class associated with high priority; such a scenario is particularly useful for auto-focusing. The movement of the various surgical instruments relative to one another may be taken into consideration. It may be taken into consideration whether the surgical instruments move toward one another or away from one another. In one variant, the fastest-moving object is classified as relevant; all other objects are classified as irrelevant; this means, in other words, that the fastest-moving object is classified in a first class; and all other surgical instruments are grouped in a second class. Optionally, a tolerance range could also be used (for example 5%). If the second-fastest-moving object moves at a similar speed (for example 95% of the movement amplitude of the fastest object), the centroid is taken. However, if an object has a significantly higher speed of movement than all other objects, only this one object is classified as relevant with high priority. A comparison could be performed between the spectra of the movement frequencies of the various surgical instruments. By way of example, it could be checked whether particular surgical instruments all have the same movement spectrum (which would be an indicator that these surgical instruments are not guided by the surgeon, but are fixed to the patient and move along with the patient's movement).

The following is a practical example: By way of example, again for the scenario illustrated in FIG. 5, the movement magnitude could thus be determined (for example based on the optical flow). The ranking of the movement magnitude is aspirator 231—bipolar coagulator 233—retractor 232. The retractor 232 thus has a significantly lower movement magnitude (relatively defined) than the aspirator 231 and the bipolar coagulator 233. It is therefore then conceivable for the aspirator 231 and the bipolar coagulator 233 to be assigned to a high-priority class, but for the retractor 232 to be assigned to a low-priority class. In such an example, it is not necessary to use fixed threshold values. This may be particularly useful if a wide variety of different scenes are to be taken into consideration and it is a priori unknown how the movement magnitude behaves in relation to the relevance of the objects. In such a case, it is helpful if, as described above, a relative criterion that is able to be applied to all scenes in a flexible and adaptive manner is used.

In a third example (again as an alternative or in addition to the examples discussed above), a machine-learned model will be used to determine the prioritization information. The machine-learned model receives the movement information as input. The machine-learned model may for example be trained to recognize movement patterns of relevant surgical instruments and to distinguish them from movement patterns of less relevant surgical instruments. The machine-learned model may then output the prioritization information with a corresponding class assignment.

A machine-learned model makes it possible to derive more complex decision rules than in the first and second examples described above. By way of example, one or more of the following factors may be considered cumulatively: distances between the instruments during movement; types of movement (for example slow versus fast), directions of movement, angle of incidence of the instruments (in order to differentiate between assistant and chief surgeon), movement patterns, etc. It may be learned to take such criteria into consideration through appropriate training. For this purpose, an expert may manually annotate corresponding input data into the machine-learned model by determining corresponding ground truths for the prioritization information.

As a general rule, the machine-learned model may also receive further inputs in addition to the input of the movement information. Examples would be for example the positioning of the two or more surgical instruments, that is to say corresponding positioning information. By way of example, a corresponding bounding box or centre localization could be transferred. A corresponding instance segmentation map could be transferred.

Information about a semantic context could also be transferred, that is to say for example an operation phase or an operation type.

A description has been given above of aspects as to how the prioritization information is determined based on the movement information. It has already been indicated above in connection with the machine-learned models that, in addition to the movement information, other data may also be taken into consideration when determining the prioritization information (this applies generally to the various examples and is not limited to the use of a machine-learned model).

By way of example, the prioritization information may additionally be determined based on the positioning of the two or more surgical instruments. It would thus be conceivable that objects that are positioned relatively centrally in the field of view tend to have a higher priority than peripherally positioned objects.

As an alternative or in addition, it would also be possible for the prioritization information to be determined based on semantic context information for a scene associated with the two or more surgical instruments. Examples of semantic context information are a type of the surgical intervention or information indicative of the phase of the surgical intervention, for example: “coagulate” or “aspirate blood”, etc.). It would also be conceivable to identify the type of surgery, that is to say for example “spinal, cranio, tumour, vascular”. By way of example, corresponding context information could be transferred to a machine-learned model as a further input. Depending on the semantic context information, for example, different lookup tables could be used to map movement information to the prioritization information. Depending on the semantic context information, different threshold values could be used for the classification in a higher-priority class or a lower-priority class based on a movement magnitude, to mention just a few examples.

As another example, it could be taken into consideration whether a particular surgical instrument is carried in the left hand or in the right hand. This may be compared with a corresponding prioritization of the hands of the surgeon in question (that is to say whether the surgeon in question is left-handed or right-handed).

In summary, in the example of FIG. 4, FIG. 5 and FIG. 6, the prioritization information is determined based on the movement information. This has the advantage of enabling particularly relevant and robust prioritization that is able to handle different types of objects. This is explained in detail below. By way of example, in U.S. Pat. No. 10,769,443 B, a distinction is made between “non-dominant” and “dominant” instruments. This distinction is made based on three classifications, namely firstly the classification of the tool type, secondly the classification as to whether the tool is assistive or non-assistive, and thirdly the classification as to the hand (right/left) in which the instrument is held. In U.S. Pat. No. 10,769,443 B, an explicit classification of the instrument type (aspirator, retractor, etc.) is thus used to decide whether an instrument is “dominant” or “non-dominant” or “assistive” or “non-assistive”. The type of the instrument has to be classified. However, in neurosurgery, there are >100 types of instruments, which are often difficult to distinguish visually. It is thus technically difficult to achieve an explicit classification. Even if a robust explicit classification of the instrument type is present, there is another problem with the solution in U.S. Pat. No. 10,769,443 B: One and the same instrument may be surgically relevant or irrelevant in some situations. By way of example, an aspirator is relevant when it is currently aspirating blood; on the other hand, it is irrelevant when the aspirator is used to merely hold back brain tissue (like a kind of hand-held “dynamic retractor”) instead of aspirating blood. This illustrates that the instrument type does not allow a direct statement about the surgical relevance of the instrument or its priority in terms of the assistance functionality. This problem is solved, in the present disclosure, by determining the prioritization information in box 3120 based on the movement information from box 3115.

The features set out above and features that are described hereinbelow may be used not only in the corresponding combinations explicitly set out, but also in further combinations or in isolation, without departing from the scope of protection of the present invention.

By way of example, a description has been given above of various aspects in connection with an assistance functionality concerning a surgical instrument. As a general rule, however, it would be conceivable to consider other types of objects, for example characteristic anatomical features of the patient.

In addition, various aspects in connection with a robotic stand have been described above. It is not absolutely necessary for the surgical microscopy system to include a robotic stand. The surgical microscopy system could also include a partially robotic stand or a manual stand.

1. A computer-implemented method for controlling a surgical microscopy system having a stand and a microscope carried by the stand,
wherein the method comprises:
driving a camera of the surgical microscopy system so as to obtain a sequence of images,
determining movement information for each of two or more objects depicted in the sequence of images, based on the sequence of images,
determining prioritization information for the two or more objects based on the movement information, and
based on the prioritization information, carrying out an assistance functionality in connection with the two or more objects.

2. The computer-implemented method according to claim 1,
wherein the prioritization information comprises a class assignment of the two or more objects at least into a first class and a second class,
wherein the assistance functionality is carried out in connection with at least one object assigned to the first class,
wherein the assistance functionality is not carried out in connection with at least one further object assigned to the second class.

3. The computer-implemented method according to claim 1,
wherein the prioritization information is determined based on the movement information using at least one predefined criterion,
wherein the at least one predefined criterion comprises a fixed movement threshold value.

4. The computer-implemented method according to claim 1,
wherein the prioritization information is obtained without any classification and, in particular, without any instance segmentation of the objects.

5. The computer-implemented method according to claim 1,
wherein the prioritization information is determined based on the movement information using at least one relative criterion that is determined based on the movement information.

6. The computer-implemented method according to claim 5,
wherein the at least one relative criterion comprises a ranking of the movement information in relation to the two or more objects.

7. The computer-implemented method according to claim 5,
wherein the at least one relative criterion comprises a relative movement of the two or more objects in relation to one another.

8. The computer-implemented method according to claim 1,
wherein the prioritization information is determined by way of a machine-learned model that receives the movement information as input.

9. The computer-implemented method according to claim 1,
wherein the prioritization information is also determined based on a positioning of the two or more objects.

10. The computer-implemented method according to claim 1,
wherein the prioritization information is also determined based on semantic context information for a scene associated with the two or more objects.

11. The computer-implemented method according to claim 1, wherein the method furthermore comprises:
receiving a user command requesting the assistance functionality in relation to a non-specific one of the two or more objects.

12. The computer-implemented method according to claim 1,
wherein the movement information comprises an optical flow between successive images of the sequence of images.

13. The computer-implemented method according to claim 1,
wherein the prioritization information is linked to a positioning of the two or more objects.

14. The computer-implemented method according to claim 1,
wherein the prioritization information comprises a segmentation map with a prioritization label.

15. The computer-implemented method according to claim 1,
wherein the prioritization information comprises a point localization with a prioritization label.

16. The computer-implemented method according to claim 1, wherein carrying out the assistance functionality comprises:
determining a target configuration of the surgical microscopy system based on the prioritization information and a positioning of the two or more objects, and
driving at least one component of the surgical microscopy system based on the target configuration.

17. The computer-implemented method according to claim 16,
wherein the target configuration of the surgical microscopy system comprises an alignment of a field of view of the microscope with respect to at least one of the two or more objects that is selected on the basis of the prioritization information.

18. The computer-implemented method according to claim 16,
wherein the target configuration of the surgical microscopy system comprises auto-focusing of the microscope with respect to one of the two or more objects that is selected on the basis of the prioritization information.

19. A data processing device for controlling a surgical microscopy system, wherein the data processing device comprises a processor that is configured to be able to load program code from a memory and to execute it, wherein the execution of the program code causes the processor to carry out the following steps:
driving a camera of the surgical microscopy system so as to obtain a sequence of images,
determining movement information for each of two or more objects depicted in the sequence of images, based on the sequence of images,
determining prioritization information for the two or more objects based on the movement information, and
based on the prioritization information, carrying out an assistance functionality in connection with the two or more objects.

20. (canceled)

21. A surgical microscopy system comprising the data processing device according to claim 19.
